(19) United States
(12) Patent Application Publication
(10) Pub. No.: US 2011/0201968 A1
(43) Pub. Date: Aug. 18, 2011

(54) Stetho-Tool

(75) Inventor: Avraham Goldstein, Flushing, NY (US)

(73) Assignee: MAGENAV, INC., Flushing, NY (US)

(21) Appl. No.: 13/092,937

(22) Filed: Apr. 23, 2011

Related U.S. Application Data
(63) Continuation-in-part of application No. 12/950,982, filed on Nov. 19, 2010.
(60) Provisional application No. 61/281,540, filed on Nov. 20, 2009.

(51) Int. Cl. A61B 7/02; F21L 4/00; F21V 33/00

(52) U.S. Cl. 600/586; 362/191; 362/253

(57) ABSTRACT

A device allows users to have a penlight handy for medical procedures. In some embodiments, the device includes a cutting tool, such as shears or a closable blade, attached to the penlight. The device may be detachable from a holster which can be secured to a stethoscope tube.

### CROSS-REFERENCE TO RELATED APPLICATIONS

This application is a continuation-in-part of U.S. patent application Ser. No. 12/950,982, filed Nov. 19, 2010, pending, which claims the benefit of priority to U.S. Provisional patent application No. 61/281,540, filed Nov. 20, 2009, both of which are herein incorporated by reference.

### BACKGROUND OF THE INVENTION

The present invention relates to a medical device and, more particularly, to a device that combines a penlight with a clip that allows the penlight to be secured to a stethoscope tube.

There are several tools consistently utilized in the field of emergency medicine to rapidly and accurately assess patients. Many medical professionals carry their stethoscope about their necks. However, other often used tools may be less readily accessible. Penlights are often used to assess a patient's pupil reactivity and shears/scissors/cutters are often used to cut away a patient's clothes, shoelaces, belt, bandages and the like. Penlights and cutters, however, are typically not carried by medical professionals and, when needed, may be difficult to find.

As can be seen, there is a need for device that allows a penlight to be carried on a stethoscope tube. There is also a need to for a device that combines a penlight and cutters that may be secured to a stethoscope tube for easy access.

### SUMMARY OF THE INVENTION

In one aspect of the present invention, a light comprises a light body; a lamp disposed on one end of the lamp body; a mounting mechanism disposed on at least a portion of one side of the lamp body, the mounting mechanism attaching the light to a stethoscope tube; and a switch for providing power to the lamp.

In another aspect of the present invention, a medical tool comprises a stethoscope; a holster removably attached to a tube of the stethoscope; a penlight/cutter combination unit removably attached to the holster, wherein the penlight/cutter combination unit includes a penlight and at least a first blade, the first blade pivotably attached to the penlight.

These and other features, aspects and advantages of the present invention will become better understood with reference to the following drawings, description and claims.

### BRIEF DESCRIPTION OF THE DRAWINGS

FIG. 1 is a perspective view of a stethoscope having a stetho-tool attached thereto;

FIG. 2 is a perspective view of a penlight/shears tool (stetho-tool) according to an exemplary embodiment of the present invention;

FIG. 3 is a perspective view of the penlight/shears tool of FIG. 2 with the shears in a closed position;

FIG. 4 is a perspective view of the penlight/shears tool of FIG. 2 positioned in a holster that is adapted to be attached to a stethoscope;

FIG. 5 is a perspective view of the holster of FIG. 4 with the penlight/shears tool removed therefrom;

FIG. 6 is a perspective view of a penlight/cutting tool according to another embodiment of the present invention;

FIG. 7 is a perspective view of a penlight/cutting tool according to another embodiment of the present invention;

FIG. 8 is a left side perspective view of a penlight according to an exemplary embodiment of the present invention;

FIG. 9 is a right side perspective view of the penlight of FIG. 8;

FIG. 10 is a perspective view of the penlight of FIG. 8 attached to a stethoscope;

FIG. 11 is a perspective view of a penlight according to another embodiment of the present invention;

FIG. 12 is a front view of the penlight of FIG. 11;

FIG. 13 is a perspective view of the penlight of FIG. 11 attached to a stethoscope; and

FIG. 14 is a perspective view of a penlight being used by a person, according to an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides a device that allows users to have a penlight handy for medical procedures. In some embodiments, the device includes a cutting tool, such as shears or a closable blade, attached to the penlight. The device may be detachable from a holster which can be secured to a stethoscope tube.

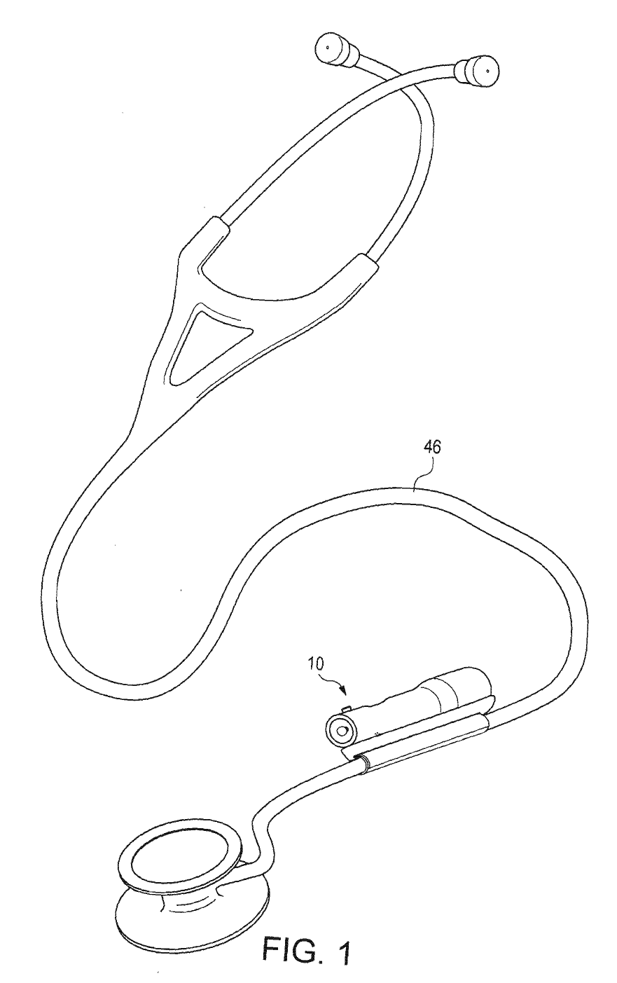

Referring to FIGS. 1 through 5, a penlight/shear tool 10 may include a penlight 12 with shears 14. The shears 14 may include a first blade 16 integrally formed into the penlight 12. A second blade 18 of the shears 14 may pivot from the penlight 12. The first blade 16 and the second blade 18 may interact, via the pivoting action of the second blade 18, to create a cutting tool. In some embodiments, the first and second blades 16, 18 may be smooth. In other embodiments, the first and second blades 16, 18 may be serrated. The second blade 18 may include a pressing surface 20 opposite a cutting surface 22 of the second blade 18. The pressing surface 20 may provide a location for a user to comfortably depress the second blade 18 onto the first blade 16. An indent 24 may be provided in the pressing surface 20.

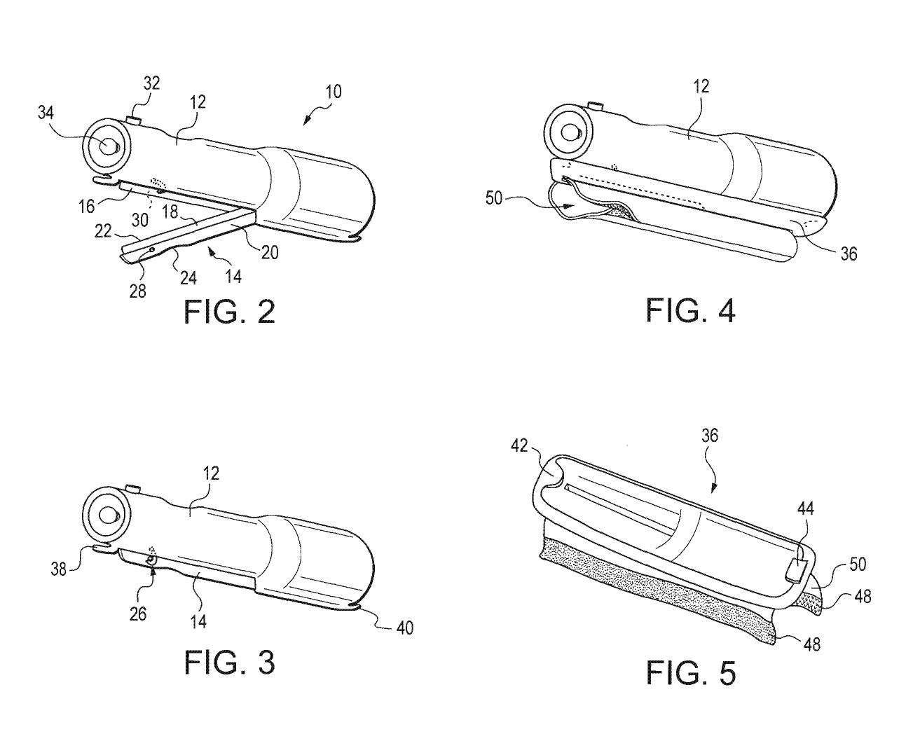

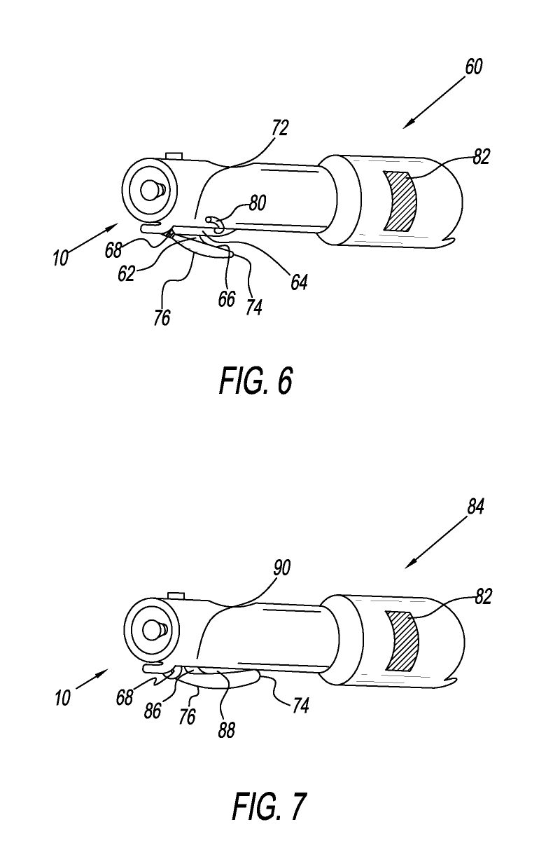

As shown in FIG. 2, the shears 14 may open such that the first and second blades 16, 18 pivot hear an end opposite the lamp. In other embodiments, the first and second blades 16, 18 may pivot at the end that the lamp is located. This alternate pivoting design may be similar to the blade design of FIGS. 6 and 7, described below.

The shears 14 may include a means for resiliently pushing open the second blade 18 from the first blade 16. For example, an internal spring may be provided to normally keep the blades 16, 18 separated. A lock 26 may be provided for locking the second blade 18 next to the first blade 16 (in a configuration similar to closed scissors, see FIG. 3). The lock 26 may be any mechanism known in the art. For example, the lock 26 may include a hole 28 in the second blade 18 that may receive a pin 30.

The tool 10 may include a push button 32 for turning on the penlight 12. Typically, the push button 32 will be a momentarily on push button, wherein the penlight 12 is lit only during the time that the push button 32 is depressed. The tool 10 may include a battery (not shown) for powering the penlight 12. The battery may be, for example, a replaceable battery. In some embodiments, the penlight 12 is powered by 1 or 2 size-AA batteries. The penlight 12 may include a lamp 34. The lamp 34 may be any known lamp that may light by the power source in the penlight 34. In some embodiments, the lamp 34 may be a light emitting diode (LED).

The tool 10 may be adapted to be secured in a holster 36. In some embodiments, the tool 10 may include front and rear tabs 38, 40 that may fit into slots 42, 44 of the holster 36. Other attachment means for removably attaching the tool 10 to the holster 36 may be within the scope of the present invention. In some embodiments, the tool 10 may fit into the holster 36 with the shears 14 being disposed between the tool 10 and the holster 36.

The holster 36 may include an attachment strap 38 for attaching the holster 36 to a stethoscope 46. In some embodiments, the attachment strap 38 may fit into a slot in the holster 36. In other embodiments, a separate attachment device may secure the attachment strap 38 to the holster 36. The attachment strap 38 may have two ends 48 descending from the holster 38. The ends may attach together by known methods. For example, the ends may attach together with a hook and loop type fastener, such as Velcro®. An inside portion 50 of the attachment strap 38 may be formed with a non-slip material, such as a thin rubber material. This non-slip material may help secure the holster 36 to the stethoscope 46 and prevent the holster 36 from sliding on the tubing of the stethoscope 46.

To use the tool 10, the holster 36 may be secured, with the attachment strap 38, to the tubing of the stethoscope 46. The tool 10 may be removably secured to the holster 36. When the tool 10 is needed (as a penlight and/or as shears), the tool 10 is simply removed from the holster 36. The holster 36 may stay in place on the stethoscope 46.

Referring now to FIG. 6, a tool 60 may be similar to the tool 10 as described above, but, in place of the shears 14, a first cutting blade 62 and a second cutting blade 64 may be disposed on the tool 60. The first cutting blade 62 may be secured to a pivoting arm 66 that may pivot about a pivot point 68 located near a front end 70 of the tool 60. The second blade 64 may be disposed on a penlight body 72. The first blade 62 and the second blade 64 may be disposed adjacent to each other. When the pivoting arm 66 is opened (as shown in FIG. 6), the tool 60 may be pulled (in a direction generally away from the direction light from the tool 60 may shine) to cut away material, bandages, seatbelts or the like. The pivoting arm 66 may have a hooked-shape with a rounded-pointed end 74. This end design may permit the pivoting arm 66 to easily be positioned under bandages and the like.

The pivoting arm 66 may include a stop mechanism (not shown) such as a protrusion, that may prevent the pivoting arm 66 from opening more than a predetermined angle 76. This angle 76 may be from about 5 degrees to about 80 degrees. Typical, this angle 76 may be from about 20 degrees to about 40 degrees.

Similar to the tool 10 of FIGS. 1 through 5, the tool 60 may include a hole 78 and a pin 80 for keeping the pivoting arm 66 in a closed position. In other embodiments, the pivoting arm 66 may be attached to a resilient member, such as a spring (not shown) for retaining the pivoting arm 66 in both an open and a closed position, similar to the mechanism for opening and closing a conventional pocket knife, except the pivoting arm 66 of the tool 10 only opens to a certain angle 76.

The tool 60 may include a thumb indentation 82. The thumb indentation 82 may help a user to hold the tool 60, especially when pulling the tube to use the cutting mechanism.

Referring now to FIG. 7, a tool 84 may include only one blade 86. The blade 86 may, in some embodiments, have a curved edge such that when material is brought to the opened blade 86, the material may slide along the curved edge, helping to cut the material. Otherwise the tool 84 may be similar to the tool 60 described above. In some embodiments, the tool 84 may include the one blade 86 on a pivoting arm 88. In other embodiments (not shown), the tool may include one blade 86 on a penlight body 90.

While the blade designs of FIGS. 6 and 7 show the blade opening from a pivot point near the lamp end of the light, the tools may also be designed to open in the opposite direction, similar to the shears described above.

In any of the above embodiments, with shears 14, with first and second blades 62, 64, or with a single blade 86, the cutting members may be made from metal, typically stainless steel, more typically 440 stainless steel. Each of the tools 10, 60, 84 described above may be used in its holster (for attachment to a stethoscope tube) or without its holster, simply as a dual purpose penlight/cutting tool.

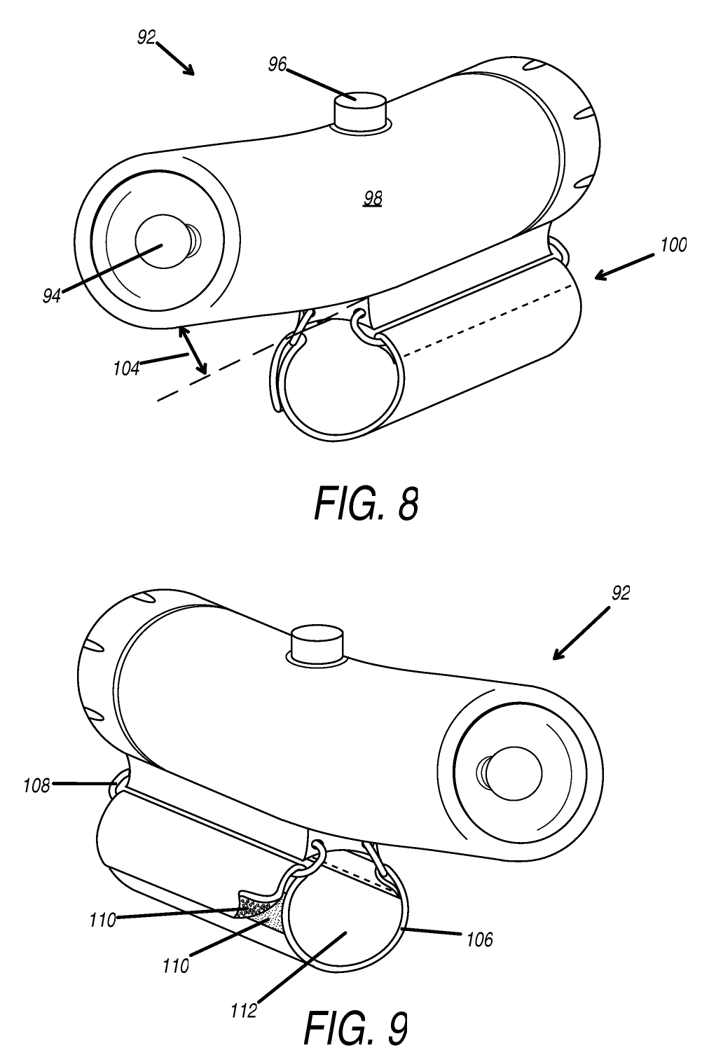

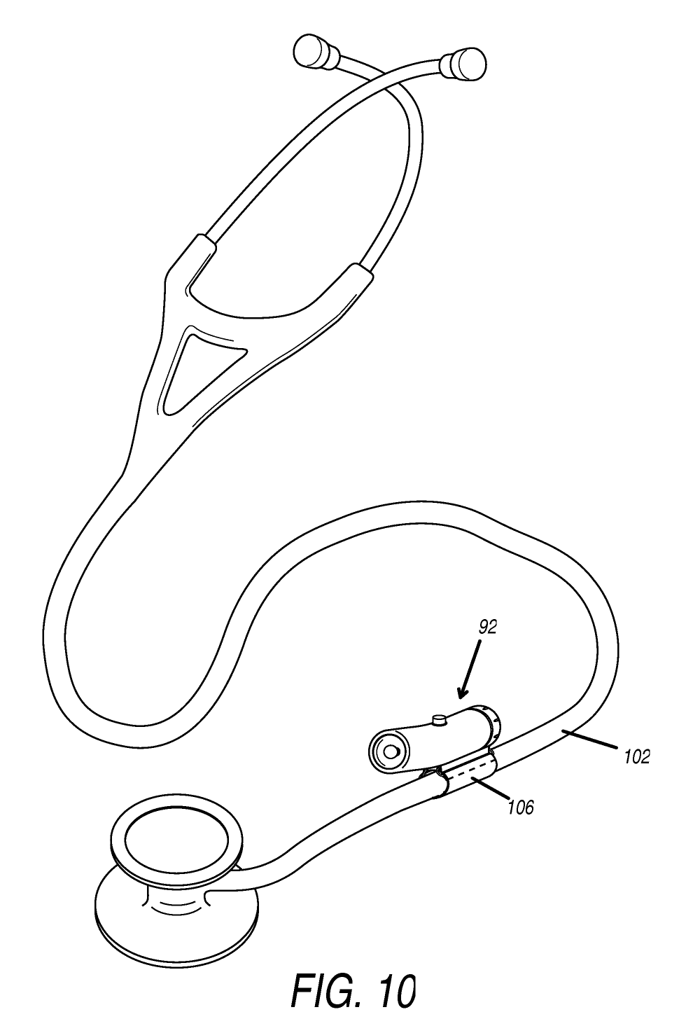

Referring now to FIGS. 8 through 10, a penlight 92 may include a lamp 94 for directing light and a switch 96 for turning on the lamp 94. The switch 96 may be a momentary ON switch, wherein power to the lamp 94 is only supplied while the switch 96 is depressed. The penlight 92 may include a mounting mechanism 100 to mount the penlight 92 onto a stethoscope tube 102. A penlight body 98 may be generally cylindrical having a slight angle 104 so that the light from the lamp 94 shines away from the stethoscope tube 102. In other embodiments, the penlight body 98 may have different shapes, such as a flattened disc shape. This angle 104 may be from about 0 to about 40 degrees, typically from about 25 to about 40 degrees, for example.

The mounting mechanism 100 may include a strap 106 adapted to be threaded through a loop 108 after the strap 106 is secured around the stethoscope tube 102. The strap 106 may include hook and loop-type fasteners, such as Velcro® for securing the strap 106 around the stethoscope tube 102. An inside surface 112 of the strap 106 may be coated with a rubber material to help prevent the penlight 92 from sliding on the stethoscope tube 102.

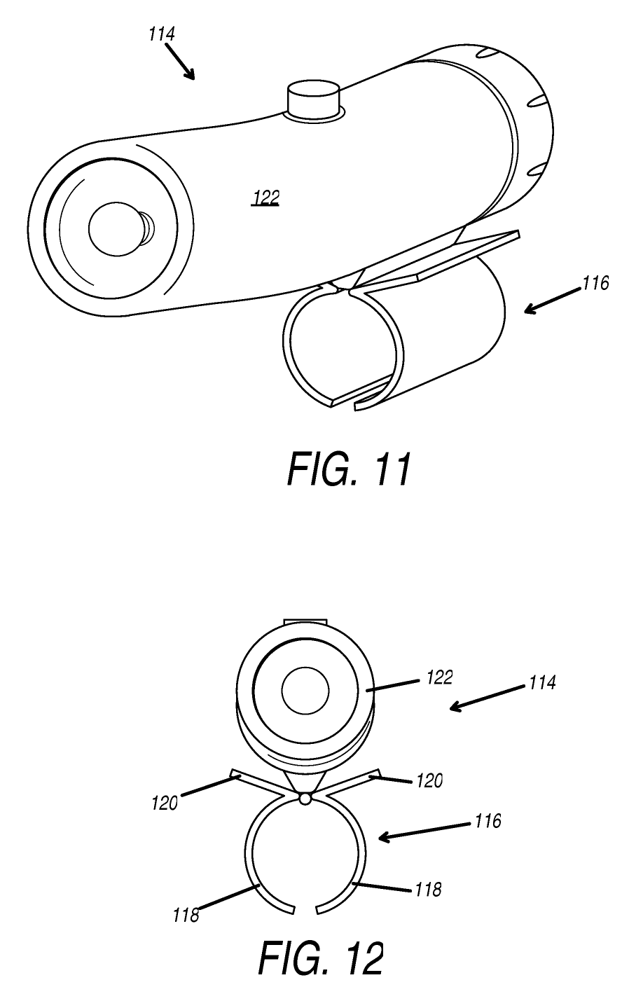

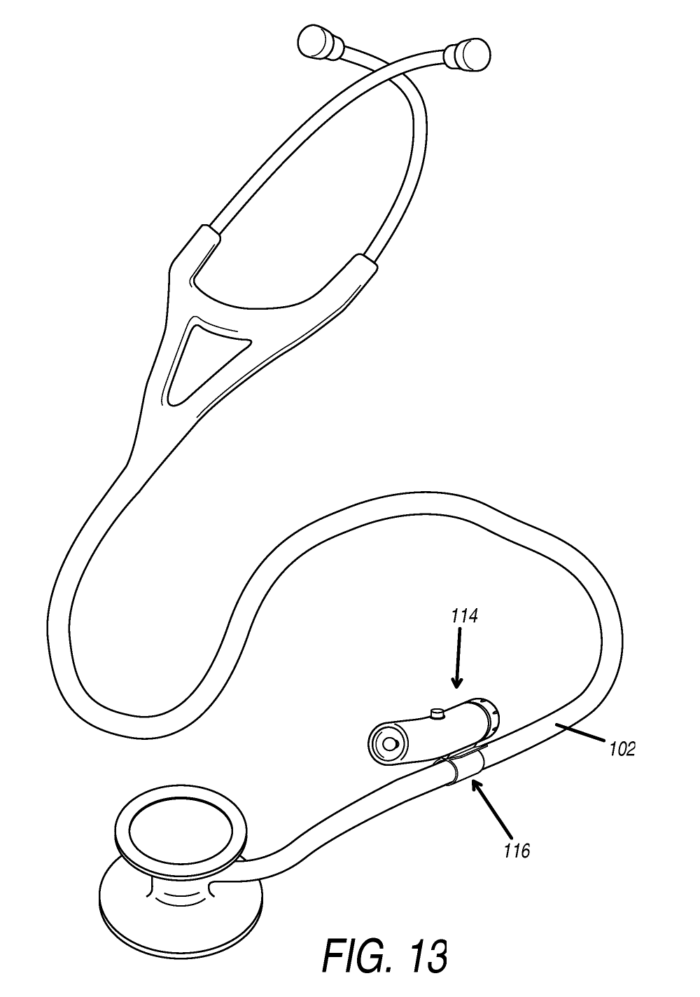

Referring to FIGS. 11 through 13, a penlight 114 maybe similar to the penlight 92 as described above, but the penlight 114 may include a spring clip 116 for attaching the penlight 114 onto the stethoscope tube 102. The spring clip 116 may be made from first and second semi-circular members 118 wherein a spring (not shown) resiliently disposes the members 118 in a closed position as shown in FIGS. 11 and 12. When arms 120 of the members 118 are pulled in toward a penlight body 122, the members 118 may separate to allow the penlight 114 to be secured to or removed from the stethoscope tube 102.

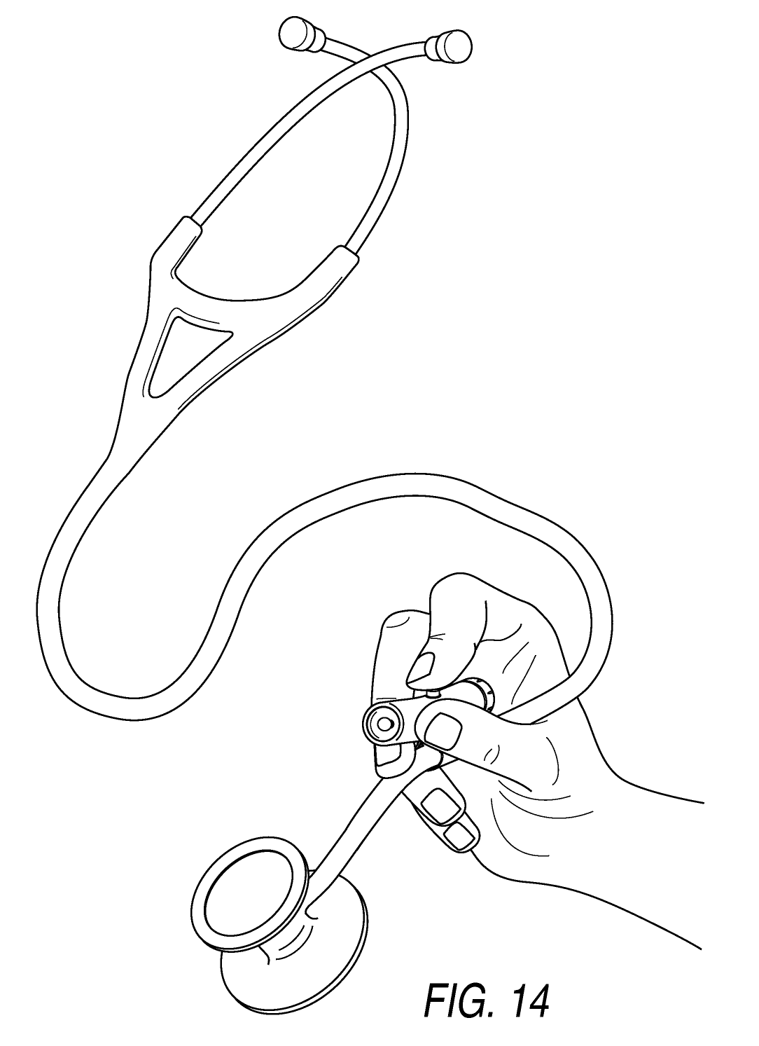

Referring to FIG. 14, to use the penlight (either penlight 92 or penlight 114, for example), the user may depress the button 96 and direct the light as needed. The angle 104 (see FIG. 8) directs the light slightly away from the stethoscope tube 102.

It should be understood, of course, that the foregoing relates to exemplary embodiments of the invention and that modifications may be made without departing from the spirit and scope of the invention as set forth in the following claims.

What is claimed is:

1. A light comprising:
a light body;
a lamp disposed on one end of the lamp body;
a mounting mechanism disposed on at least a portion of one side of the lamp body, the mounting mechanism attaching the light to a stethoscope tube; and
a switch for providing power to the lamp.

2. The light of claim 1, wherein the lamp body has a cylindrical shape with a bend, the bend causing the lamp to shine light at an angle relative to a plane of the mounting mechanism.

3. The light of claim 2, wherein the angle is from about 25 to about 40 degrees.

4. The light of claim 1, wherein the mounting mechanism includes:
a strap adapted to wrap around the stethoscope tube;
a loop adapted to receive an end of strap; and
hook and loop fasteners on the end of the strap.

5. The light of claim 4, wherein an inside of the strap is coated to prevent the light from sliding along the stethoscope tube when the light is secured thereto.

6. The light of claim 1, where the mounting mechanism includes a spring clip.

7. The light of claim 1, further comprising:
shears attached to the penlight;
a holster for removably holding the penlight and shears;
a strap adapted to secure the holster to a stethoscope.

8. The light of claim 7, wherein a first blade of the shears is affixed to the penlight and the second blade of the shears pivots relative to the first blade.

9. The light of claim 7, further comprising a button, wherein the penlight has a lamp that is turned on during depression of the button.

10. The light of claim 7, further comprising a slip resistant coating on an inside surface of the strap, the slip resistant coating adapted to prevent slipping of the holster when attached to the stethoscope.

11. The light of claim 8, further comprising means for resiliently opening the second blade from the first blade.

12. The light of claim 11, wherein the means includes an internal spring.

13. The light of claim 11, further comprising a latch for holding the second blade closed against the first blade.

14. The light of claim 7, further comprising first and second tabs formed on ends of the penlight.

15. The light of claim 14, further comprising first and second slots on the holster, wherein the first and second tabs are adapted to removably fit into the first and second slots to secure the penlight to the holster.

16. The light of claim 7, further comprising a pressing surface disposed on the second blade, opposite a cutting surface of the second blade.

17. The light of claim 16, further comprising an indent formed in the pressing surface.

18. The light of claim 1, further comprising:
a first blade attached to a pivoting arm, wherein the pivoting arm pivots away from the light body at a front end thereof.

19. The light of claim 18, further comprising a second blade disposed on the light body, the second blade being adjacent to the first blade.

20. The light of claim 18, wherein the pivoting arm is hooked shape with a rounded pointed end.

21. A medical tool comprising:
a stethoscope;
a holster removably attached to a tube of the stethoscope;
a penlight/cutter combination unit removably attached to the holster, wherein the penlight/cutter combination unit includes a penlight and at least a first blade, the first blade pivotably attached to the penlight.

22. A light/cutting combination tool comprising:
a light body;
a lamp disposed on one end of the lamp body;
a cutting tool pivotably disposed on at least a portion of one side of the light body; and
a switch for providing power to the lamp.
